(19) United States
(12) Patent Application Publication
(10) Pub. No.: US 2003/0149456 A1
(43) Pub. Date: Aug. 7, 2003

(54) Multi-electrode cardiac lead adapter with multiplexer

(76) Inventors: William B. Rottenberg, Durango, CO (US); John K. Prentice, Durango, CO (US); John A. Schmidt, Durango, CO (US)

Correspondence Address: GOTTLIEB RACKMAN & REISMAN PC, 270 MADISON AVENUE, 8TH FLOOR, NEW YORK, NY, 100160601

(21) Appl. No.: 10/062,138

(22) Filed: Feb. 1, 2002

(51) Int. Cl. A61N 1/36

(52) U.S. Cl. 607/37

(57) ABSTRACT

An adapter, which allows the use of a complex multi-electrode pacing and sensing cardiac lead with a conventional one-channel cardiac pacing pulse generator has a connector that attaches to the multi-electrode cardiac lead, a multiplexer to select which of the electrodes of the multi-electrode cardiac lead are to be connected to the one channel cardiac pacing pulse generator, and a connector to connect the adapter to a connector such as the industry standard IS-1 connector. New therapies are developing requirements for both optimum-site pacing (rather than the traditional ventricular apex) and multi-site pacing to improve cardiac hemodynamics. These therapies require custom pulse generators that are capable of connecting to the multiple-electrode configurations required. This adapter allows a conventional one-channel cardiac pacing pulse generator to be connected to a multi-electrode cardiac lead and steers the output pulse to the desired set of electrodes.

### BACKGROUND OF THE INVENTION

1. Field of the Invention

This invention relates to cardiac pacing systems. More particularly, this invention relates to adapters that provide connection between multi-electrode cardiac leads placed in contact with a patient's heart and a standard cardiac stimulator.

2. Description of Related Art

Cardiac stimulation systems consist of two basic components, a pacing device commonly referred to as a pacemaker and a cardiac pacing lead. The pacemaker monitors the intrinsic electrical signals of the heart to detect arrhythmia and generates therapeutic electrical signals when required. In this application the term pacemaker is used generically to cover any implantable cardiac stimulator. A cardiac pacing lead is an insulated wire that carries the stimulation signal from the pacemaker into the chambers of the heart. A cardiac pacing lead may have a fixation mechanism, near its distal end to hold it in place. The lead has at least one electrode adapted to sense and/or to deliver signals from the pacemaker to the heart tissue.

A connector is formed at the proximal end of a cardiac pacing lead opposite the electrode. The connector has contacts that are inserted into a connector block of the pacemaker usually referred to as a header. The connector of a cardiac pacing lead and the connector block generally conform to the International Standards Organization standard ISO 5841 or the European standard EN 50077 1992 and commonly referred to as the IS-1 standard. The IS-1 standard defines the electrical and mechanical characteristics of the lead connection to the pacemaker.

Typically, cardiac stimulation requires electrodes attached or at least in contact with the myocardium to stimulate the cardiac muscle. Earlier stimulators utilized electrodes implanted into epicardium, primarily because the technology had not been developed to implant a lead transvenously. With the development of the transvenous endocardial electrode, epicardial leads decreased due to the increased invasiveness of the procedure.

The original pacemakers stimulated only one chamber of the heart, the atrium or the ventricle. Modern devices have the capability of stimulating both chambers, thereby restoring the natural Atrial-Ventricular synchrony.

Typical endocardial pacemaker leads are implanted into the right atrium, right ventricle, the coronary sinus, or the great cardiac vein and have a limited number of sensing and stimulating electrodes located in the heart chamber or coronary vasculature. For conventional bradycardia pacing and for conventional tachycardia pacing and defibrillation, the distal tip of the right ventricular leads have traditionally been implanted into the apex of the ventricle. However, numerous clinical studies have documented that left ventricular hemodynamics is compromised by this pacing location. Better hemodynamics can be achieved by implanting the lead tip somewhere on the septal wall or in the right ventricular outflow tract.

In biventricular pacing of the left ventricular free wall, a lead is inserted through the coronary sinus into the great cardiac vein. This makes it possible to pace the left ventricle epicardially to improve left ventricular cardiac performance in congestive heart failure patients. However, this type of stimulation is extremely sensitive to the location of the pacing electrode(s) in the great cardiac vein. In both conventional right ventricular and great cardiac vein stimulation, it is required to reposition the lead many times before finding the optimal stimulation site. In other words, a trial and error process is used requiring repeatedly moving the lead tip, looking at some measure of cardiac output (for example, blood pressure, QRS duration, or flow velocity in the left ventricular outflow tract) until the best site is found to optimize cardiac output. This can be an extremely time consuming process. More importantly it increases both cost and patient risk.

In general, various types of cardiac leads containing electrodes have been used to perform endocardial procedures for treatment and diagnosis of cardiac related problems, such as the stimulation cardiac lead of U.S. Pat. No. 3,825,015 (Berkovits), the flow directed cardiac lead of U.S. Pat. No. 3,995,623 (Blake et. al.), the multi-contact plunge electrode of U.S. Pat. No. 4,172,451 (Kline), the defibrillating cardiac lead of U.S. Pat. No. 5,545,205 (Schulte et al.), the implantation cardiac lead of U.S. Pat. No. 5,800,498 (Obino et al.) and a cardiac pacing lead delivery cardiac lead of U.S. Pat. No. 6,055,457 (Bonner). U.S. Pat. No. 4,603,696 (Cross) shows a lead diameter for a multi-electrode lead. U.S. Pat. No. 6,295,475 (Morgan) shows an adapter for a multi-electrode lead.

Multi-electrode cardiac leads have been used to map cardiac electrical activity. This mapping procedure is useful for the detection and treatment of conduction abnormalities and heart tissue deficiencies. Some cardiac mapping procedures are described in the article entitled “Techniques of Intraoperative Electrophysiologic Mapping” in the American Journal of Cardiology, by John J. Gallagher, et al. which appeared in Volume 49 pages 221-240 January of 1982.

During a typical mapping procedure, a cardiac map is generated by recording the electric signals from the heart and depicting them spatially as a function of time. A multi-electrode cardiac lead is inserted into a chamber of the heart—to measure signals directly by contact with the inside walls of the chamber. Accordingly, the number and placement of electrodes on or within the cardiac lead is an important design consideration for maximizing effectiveness and efficiency for this internal procedure.

Several types of multi-electrode cardiac leads have been used to generate cardiac maps. For example, U.S. Pat. No. 4,573,473 (Hess) teaches a cardiac lead with four electrode contacts on a flat planar surface. U.S. Pat. No. 4,522,212 (Gelinas et al.) teaches a cardiac lead with three or more separated flexible leg electrodes. U.S. Pat. No. 4,699,147 (Chilson) and U.S. Pat. No. 5,471,982 (Edwards) define cardiac leads with flexible electrodes that form a basket when extended.

Multi-electrode leads have also been used for ablation.

The concept of lead adapters is not new. In early pacemakers, the connection mechanisms varied among manufacturers and even within a manufacturer's line as technology developed. When the pulse generator required replacement, either due to malfunction, clinical considerations or battery depletion, it was not uncommon to have an incompatibility between the old lead that may still be viable and the new pulse generator. Since the distal end lead becomes strongly encapsulated shortly after implantation in the heart, removal of the old lead is impractical and often dangerous to the patient. This leaves the clinician with the choice of either adapting the old lead to the new pulse generator or abandoning the old lead, leaving it in place and implanting a new lead. Implanting a new lead has the disadvantage of adding additional hardware in the patient's heart, with the associated risks, as well as the risk and complexity of the new lead implantation. It is far more desirable to reuse the original lead, so adapters for this purpose became common. These adapters did not provide any therapeutic improvement or increased capability, but simply allowed the different connector mechanisms between the new pulse generator and the older lead to be used together. They consisted of a short wire with a connector on one end compatible with the new pulse generator and a connector on the other end that was compatible with the original lead.

U.S. Pat. No. 4,628,934 (Pohndorf) describes an electronic electrode switching/selection circuit that minimizes the number of feed-through openings from a case to the neck needed to connect with pacing lead electrodes that will be actively used during operation.

U.S. Pat. No. 5,222,506 (Patrick et al.) illustrates an adapter for switching the conductors of bipolar pacemaker leads so that the stimulating and return conductors are reversed.

U.S. Pat. No. 5,507,787 (Borghi) describes an adapter that includes anew conductor that is passed through the length of an existing lead, thereby providing another conductive path if the original lead has a wire failure.

U.S. Pat. No. 5,797,970 (Pouvreau) and U.S. Pat. No. 4,289,134 (Bernstein) describe methods for delivering stimulation to the heart through a series of leads utilizing conventional pacing technology and leads with one stimulation site per lead.

U.S. Pat. No. 4,740,170 (Lee) and U.S. Pat. No. 4,583,543 (Peers-Trevarton) describe an upsizing adapter that is used to enlarge a smaller lead connector to fit into a larger pulse generator connector hole.

U.S. Pat. No. 5,679,026 (Fain) illustrates a rigid adapter that attaches to the pulse generator and provides connector ports for a cardiac pacing lead.

### OBJECTIVES AND SUMMARY OF THE INVENTION

An objective of this invention is to provide an adapter to connect a multi-electrode cardiac lead to a conventional pacemaker.

Further, another objective of this invention is to provide an adapter for a multi-electrode cardiac lead to be attached to a conventional pacemaker where one grouping of the electrodes of lead providing connections for sensing and a second set of electrodes of the multi-electrode cardiac lead providing connections for pacing or other cardiac stimulation.

Another objective of this invention is to provide a method for selecting electrodes of a multi-electrode cardiac lead are that to be connected through an adapter to a single or dual leaded conventional pacemaker.

To accomplish these and other objectives an adapter is connected between a multi-electrode cardiac lead and a pacemaker with specific electrodes of the lead being connected to specific contacts of the pacemaker. The multi-electrode cardiac lead has a distal end, which includes a plurality of electrodes placed in contact with a heart and a proximal end having plurality of lead terminals. Each lead terminal is connected to one electrode by a wire extending through the lead.

The adapter has a multiplexer connected between a first and a second connector. The first connector receives the plurality of lead terminals. The multiplexer has an input connected to the first connector, a selector connected to the input to select a group of electrodes of the multi-electrode cardiac lead, and an output to transfer sensing signals and pacing signals to or from a second connector. The second connector has less contacts than the number of lead terminals attached to the first connector.

The selector of the multiplexer is formed of a plurality of links. Each link is connected between each terminal, and a contact of the second connector. A link programmer chooses the links required to connect at least some of to the contacts of the terminals to the contacts of the second connector. In one embodiment the links are metallic breakable links which open a circuit in response to a signal from the programmer. In an alternate embodiment, the links are fusible links, which close an electrical path when the programmer applies an electrical signal thereto.

In a second embodiment, the selector is formed of a plurality of electronic switches. Each switch has a first switch terminal connected to one lead terminal, a second switch terminal connected to one contact of the second connector, and a control terminal that receives a control signal that causes the first switch terminal to be connected selectively to the second switch terminal. The switches can be formed of field effect transistor (FET) pass gates or transmission gates.

The adapter further has a control circuit connected to the control terminal of each switch to provide the control signals, thereby activating some of the switches. A programming interface is connected to the control circuit to provide an encoded programming signal indicating which of the multiple electrodes are to be connected to the contacts of the second connector. The control circuit decodes the encoded programming signal to form the control signal. A radio frequency receiver is attached to the control circuit to receive the encoded programming signal as a radio frequency transmission from a radio frequency transmitter remote from the heart and the cardiac pacing device.

The adapter may have a power conversion circuit connected to the contacts of the second connector and the multiplexer to convert a portion of the energy present in a pacing pulse provided by the cardiac pacing device to a voltage to power the multiplexer. The power conversion circuit may have a battery connected to provide backup power if the cardiac pacing device does not provide the pacing pulses for a while. The power conversion circuit has a capacitor in communication with a pacing contact to receive and retain the energy from the pacing pulse. A first diode is connected between the pacing contact of the second connector and the multiplexer circuit to prevent the voltage of the battery from being fed to the cardiac pacing device, while allowing the pacing pulse to be received by the capacitor. A second diode connected between the battery and the multiplexer to prevent the pacing pulse from interacting with the battery.

Instead of a single multiplexer, the adapter may have a separate pacing multiplexer and a sensing multiplexer. The pacing multiplexer has an input connected to the first connector, a selector connected to the input to chose a pacing group of electrodes, and a pace output. The sensing multiplexer has an input connected to the first connector, a selector connected to the input to chose a sensing group of electrodes of the multi-electrode cardiac lead, and a sense output. The sense and pace outputs are connected to the traditional sense and pace terminals of a standard pacemaker.

A pacing control circuit is connected to the pacing multiplexer and the contacts of the second connector to sense the presence of a pacing pulse from the cardiac pacing device to activate the pacing multiplexer to connect the pacing group of electrodes to the contacts of the second connector.

A sensing control circuit is connected to the sensing multiplexer and the contacts of the second connector to sense the presence of the pacing pulse from the cardiac pacing device to deactivate the first multiplexer and activate the second multiplexed to connect the second group of electrodes to the contacts of the second connector.

In this manner, the sensing multiplexer maintains the sensory electrodes connected to the pacemaker except during a pacing pulse.

### BRIEF DESCRIPTION OF THE DRAWINGS

FIG. 1a. is a diagram of cardiac pacing system of this invention having an adapter for an endocardial multi-electrode cardiac lead to be attached to a conventional pacemaker;

FIG. 1b. is a diagram of cardiac pacing system of this invention having an adapter for an epicardial multi-electrode cardiac lead to be attached to a conventional pacemaker;

FIG. 2. is a block diagram of an adapter constructed in accordance with this invention;

FIG. 3a. is a block diagram of the adapter of FIG. 2 with a multiplexer;

FIGS. 3a and 3c show plan views of a breakable and a fusible link, respectively, for the multiplexer of FIG. 3a;

FIG. 4 is a block diagram of the multiplexer with a programmer;

FIG. 5. is a schematic diagram of a remotely programmable multiplexer for the adapter of FIG. 3a;

FIG. 6. is a schematic diagram for a power conversion circuit for the adapter of FIG. 3a;

FIG. 7. is a schematic diagram for an adapter with control circuit;

FIG. 8. is a schematic diagram of a differential pacing and sensing control circuit for the adapter of FIG. 1;

FIG. 9 is a flow chart for a method of selecting the electrodes of a multi-electrode cardiac lead connected to the adapter of FIGS. 1-8;

FIG. 10 is a flow chart for testing for the electrodes; and

FIG. 11 is a flow chart for testing for the electrodes of the multi-electrode sensor having the minimum magnitude for a pacing signal.

### DETAILED DESCRIPTION OF THE INVENTION

U.S. patent application Ser. No. 09/761,333 filed Jan. 18, 2001 assigned to the same assignee as this invention and entitled Cardiac Electrode Catheter and Method of Manufacturing same now______, incorporated herein by reference describes an endocardial lead having multiple electrodes that can be deployed in a heart chamber or coronary vasculature. The electrodes are electrically isolated so that they can function independently. Different embodiments of this cardiac lead can be placed into the great cardiac vein, in the right atrium, and the right ventricle. In the right atrium or ventricle, the cardiac lead can be deployed so that electrodes positioned throughout the heart chamber, including the septal wall and the right ventricular outflow tract. In the great cardiac vein, multiple electrodes can be deployed along a significant length of the vasculature.

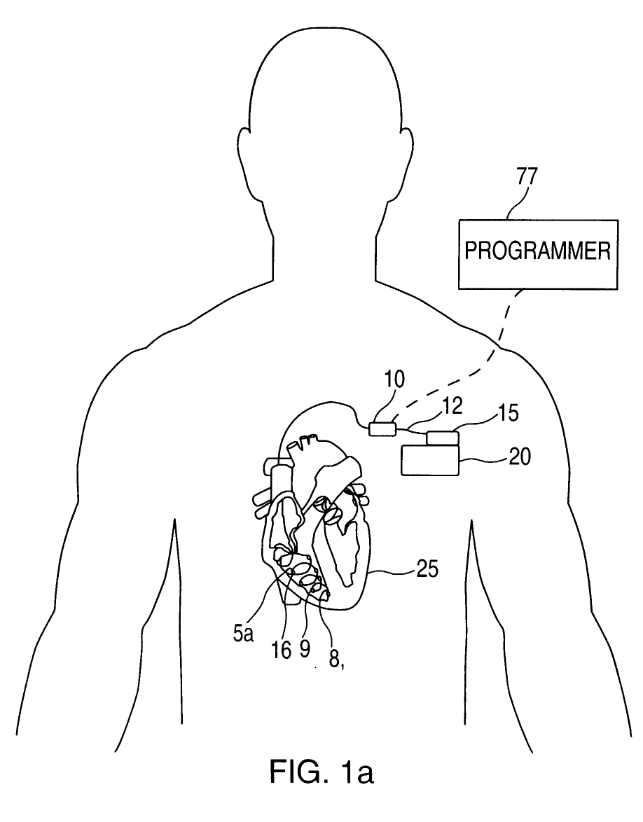

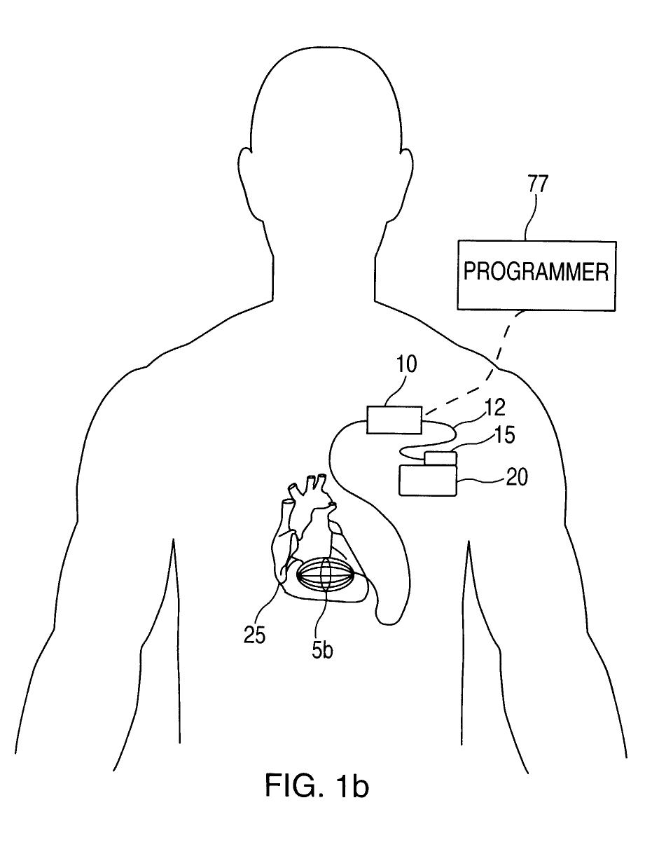

The adapter of this invention allows the terminals of a proximal end of a multi-electrode cardiac lead to be connected to the connectors of any currently marketed pacemaker or other pulse generator conforming to the IS-1 standard. Refer now to FIG. 1a for an overview of the cardiac pacing system of this invention, consisting of a lead 5a, a pacemaker 20 with a header or IS-1 connector 15 and an adapter 10. In FIG. 1a the distal end of the endocardial multi-electrode cardiac lead 5a is implanted within the heart 25 as described above. The proximal end of the multi-electrode cardiac lead 5a is coupled to the adapter 10. The adapter 10 has circuitry that selects which electrodes of the lead 5a are connected electrically to the pacemaker 20.

In FIG. 1b, the distal end of epicardial multi-electrode cardiac lead 5b is placed on the exterior surface of the heart 25. The proximal end of the epicardial multi-electrode cardiac lead 56 is coupled to adapter 10 as described above in FIG. 1a. Further, as described in FIG. 1a, the adapter 10 has circuitry to select which of the electrodes of the multi-electrode cardiac lead 5a are connected to pacemaker 20.

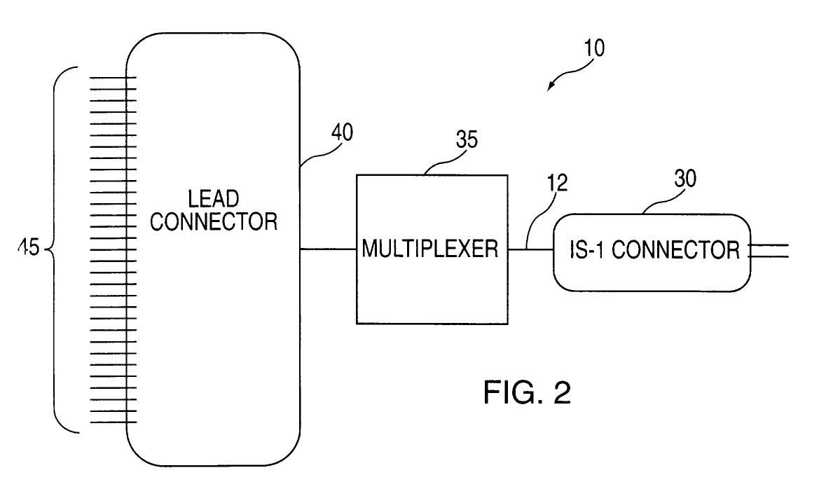

As shown in both FIGS. 1a and 1b, adapter 10 is connected to the IS-1 type connector 15 of the cardiac pacing pulse generator 20 through a multi-conducting wire 12. The general structure of the adapter 10 of this invention is shown in FIG. 2. The adapter 10 includes an IS-1 compatible connector 30 that connects to the pacing pulse generator 20. The adapter 10 also has a lead 5 (Numeral 5 is used to refer collectively to leads 5a and 5b) through the terminals 45 of the multi-electrode cardiac leads. The multiplexer 35 contains a connection matrix (discussed in detail below) that makes the required connections between the IS-1 connector 30 and the lead connector 40.

The adapter 10 can be customized for each patient or for each pacemaker using an external programming device. For example, if it is determined that multi-site pacing from electrodes 2, 9, and 16 is needed within lead 5 (shown in FIG. 1a), the appropriate connections will be made by the multiplexer 35.

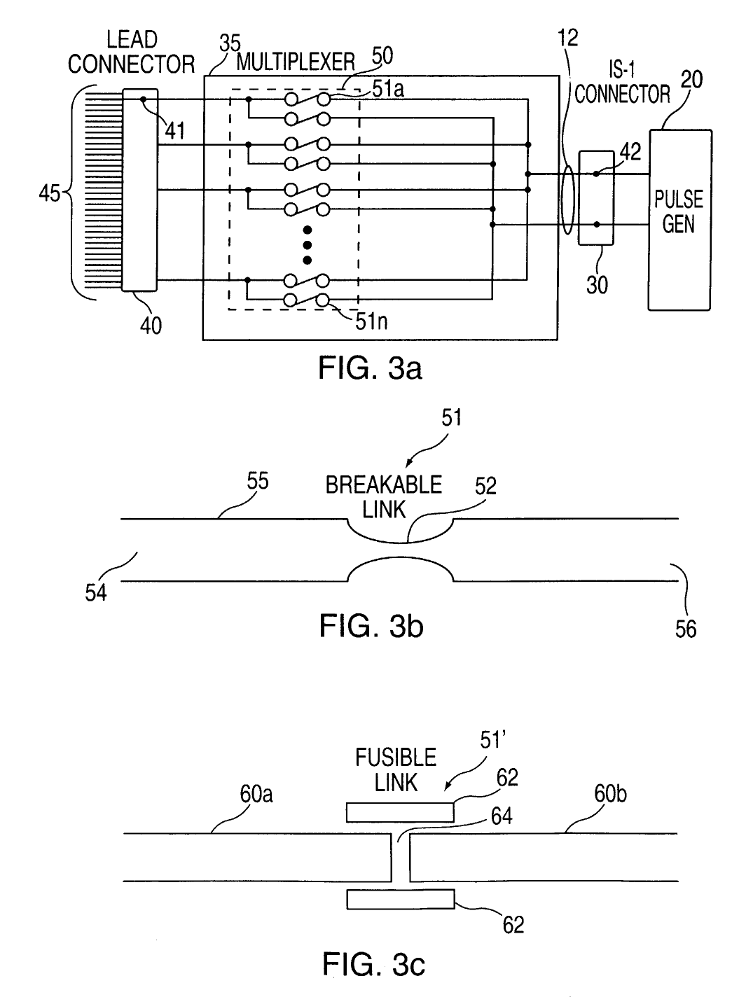

Refer now to FIG. 3a the multiplexer 35 includes a bank of links 50. The bank consists of link 51a, . . . , 51n each of which is connected between one lead terminal of the multi-lead connector 40 such as 41 and one of the contacts of the IS-1 connector 30 such as 42. The links of bank 50 can be breakable or fusible links.

FIG. 3b illustrates a typical breakable link 51 for the bank 50. The link 51 is formed as a metal conductor 55 deposited on a substrate. Alternatively, the link 51 could be formed without a substrate. The metal conductor 55 has a thinned region 52. The external programmer is attached to the ends 54 and 56 of the metal conductor 55 through connections 30 and 40. A current is forced through the metal conductor 55 until the current density in the thinned region 52 of the metal conductor 55 is sufficient to melt it and the link 51 is opened. This is a phenomenon well known in the art and not discussed further.

If the links 50 of FIG. 3a are a breakable type, an external programmer is used to break all the links of bank 50 that are not required leaving only the required link closed.

FIG. 3c illustrates a typical fusible link 51′. The link 51′ is formed of two metal conductors separated by a dielectric material 64. The dielectric material may be air, a polymeric insulator, silicon dioxide, or other known insulator. Metal conductors 62 are placed in close proximity to the separating dielectric material 64 and the ends of the two metal conductors 60a and 60b. The programmer is attached to the metal conductors 60a and 60b through connectors 30, 40. The programmer (not shown) applies a sufficiently high voltage between the metal conductors such that the separating dielectric material breaks down and a conducting plasma is formed. The heat of the plasma melts the metal conductors 62 and they fuse to form a bridge (not shown) to the metal conductors 60a and 60b. The metal conductors 62 generally are formed of a metal having a low melting point to allow the formation of the bridge at a relatively low temperature. The lower temperature should be much less than the melting point of the metal conductors 60a and 60b thus allowing fusing of the link with no degradation of the metal conductors 60a and 60b. Again, this process is well known and will not be described in more detail. For this embodiment, only the required links are fused.

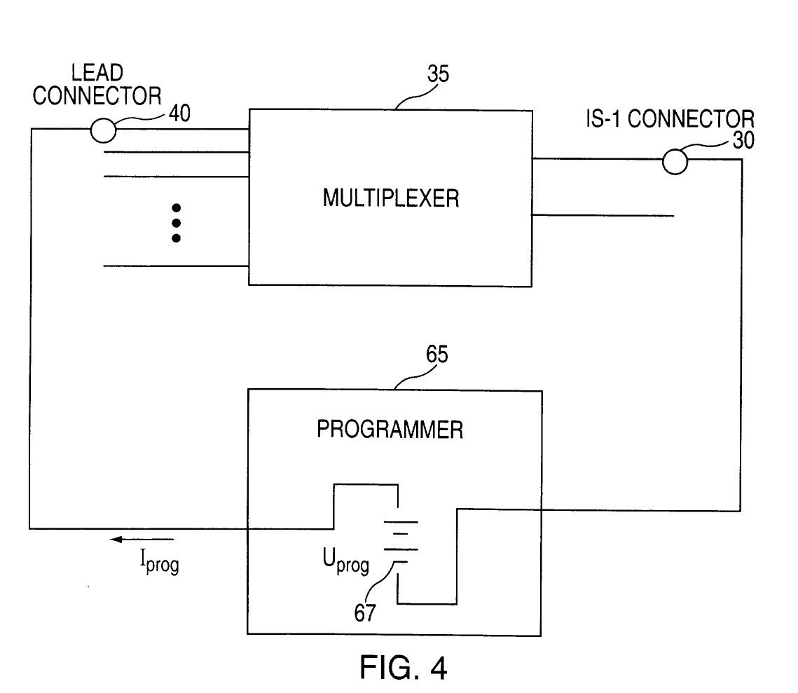

The external programmer 65, as shown in FIG. 4, has a power source 67 that provides the programming voltage (Vprog) and the programming current (Iprog). When a link 51a, . . . , 51n of FIG. 3a is to be broken or fused, the external programmer 65 is connected to one terminal of the lead connector 40 and to one contact of the IS-1 connector 30. If the link 51 of FIG. 3a is to be opened, the programming current Iprog is set to the level that allows the thinned region 52 of FIG. 3b to melt. Alternately, if the link 51′ of FIG. 3b is to be fused, the voltage Vprog is set such that the separating dielectric 64 of FIG. 3c breaks down causing a plasma which melts the metal conductors 62 of FIG. 3c to bridge the metal conductors 60a and 60b as described. The programmer 65 steps through each of the links of bank 50 and opens or closes them as required. Importantly, once a link is opened or closed, it remains in that state and the process cannot be reversed.

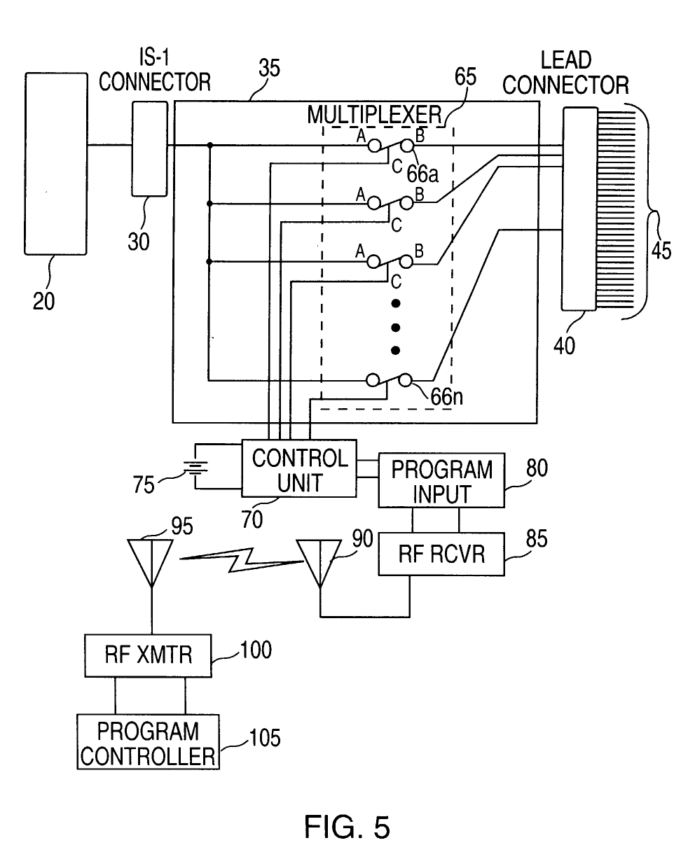

Refer now to FIG. 5 for discussion of a second embodiment of the adapter of this invention. In the second embodiment, the multiplexer is formed of a bank 65 of electronic switches. Each switch 66a, . . . , 66n of bank 65 has a first switch terminal A connected to one of the contacts of the IS-1 connector 30 and a second switch terminal B connected to one lead terminal 45 of the lead connector 40. Further, each switch 66a-n has a control terminal C connected to the control circuit 70. The control circuit 70 provides a control signal to selectively open or close switches 66a-n as required.

A program input circuit 80 is connected to the control circuit 70 the program input circuit 80 and receives an encoded programming signal. The program-input circuit 80 decodes the encoded programming signal to define the control signal to the respective switches. The program-input circuit 80 senses the control signal to the control circuit 70. The control circuit 70 then routes the control signal to the control terminal C of the desired switches 66a, . . . , 66n.

In a preferred implementation of the second embodiment of the adapter of this invention, the program input 80 is connected to a radio frequency (RF) receiver 85. The RF receiver 85 is connected to a receiving antenna 90. The receiving antenna 90 receives a radio transmission from the transmitting antenna 95. The transmitting antenna 95 is connected to the RF transmitter 100, which is connected to the program controller 105.

Upon selection of the desired group of electrodes of the multi-electrodes cardiac lead, the program controller 105 creates the encoded program signal. The program controller 105 transfers the encoded program signal to the RF transmitter, where it modulates the RF transmission. The RF transmission modulated with the encoded program signal is transferred to the transmitting antenna 95 for transmission to the receiving antenna and then to the RF receiver 85. The RF receiver 85 then demodulates the RF transmission to extract the encoded program signal. The encoded program signal is then transferred to the program input circuit 85.

The methods and techniques for programming cardiac pacing systems is well known in the art and are not discussed further.

A power source 75 is connected to provide voltage to the control circuit 70, the multiplexer 35, the program input circuit 80 and the RF receiver 85. The power source could be a battery included within the adapter.

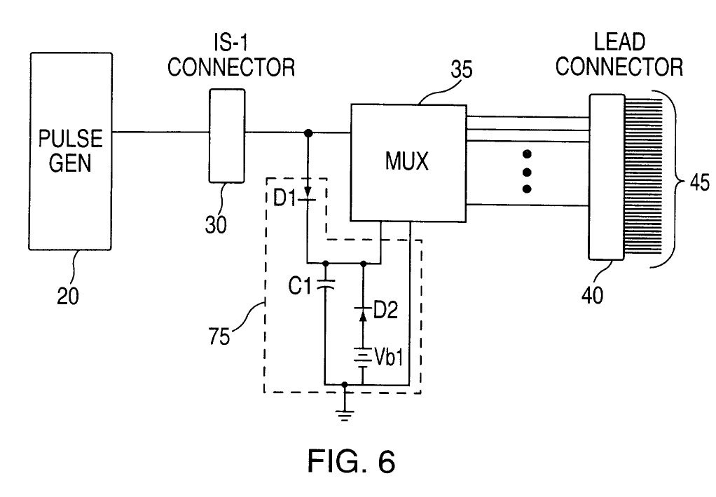

In an alternate implementation of the second embodiment of the adapter of this invention, the power source 75 has a power conversion unit connected through the IS-1 connector 30 to the pulse generator 20. The power conversion circuit captures a portion of the energy present in the stimulation signal provided by the pulse generator 20 and converts the energy to a voltage to power the circuit incorporated in the adapter 10. The power conversion circuit shown in FIG. 6 has a capacitor C1, which is charged during the active period of the pulse. The capacitor C1 is connected to act as a voltage source to power the multiplexer circuit 35. A diode D1 is connected between the capacitor C1 and the contact of the IS-1 connector 30 to prevent the charge present on the capacitor C1 from being transferred back to the contacts of the IS-1 connector 20 when the pulse is not active.

The power conversion circuit 75, additionally, has a rechargeable battery Vb1 which acts as a voltage source if the pacing signal does not provide sufficient energy to keep the capacitor C1 charged adequately to power the multiplexer circuit 35. The diode D2 is connected between the capacitor C1 and the battery Vb1 to prevent the charge present on the capacitor C1 from trying to charge the battery Vb1. Capacitor C1 can be connected through appropriate diodes to a plurality stimulation wire from pulse generator 20.

As described above, multi-focal pacing or optimal site pacing can be achieved by having one electrode or group of electrodes of the multi-electrode cardiac lead designated for transmission of the stimulation signal and another electrodes or group electrodes of the multi-electrode cardiac lead to provide sense points for sensing the heart activity. This requires that different sets of electrodes of the multi-electrode cardiac lead be connected through the adapter to the stimulation pulse generator during the period that the stimulation signal is active than when stimulation signal is inactive and the pacemaker is sensing the heart activity.

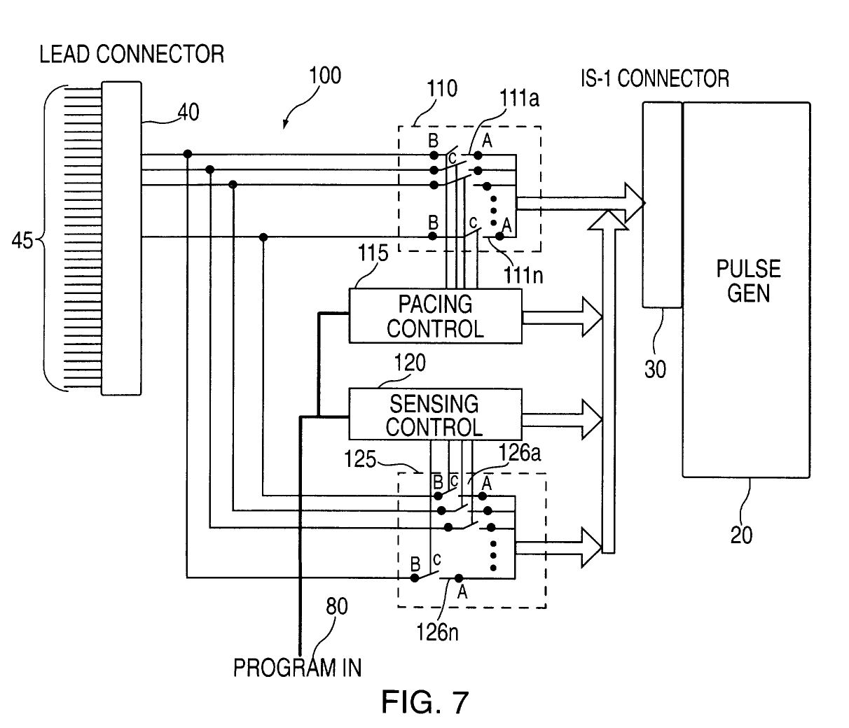

FIG. 7 illustrates a third embodiment of the adapter of this invention where a pacing set of electrodes is coupled to the pulse generator during the time the pacing signal is active and a sensing set of electrodes is coupled to the pulse generator during the time that the pacing signal is inactive.

The adapter 100 of this embodiment has two multiplexers, a pacing multiplexer 110 and a sensing multiplexer 125. The pacing multiplexer 110 and the sensing multiplexer 125 are formed of electronic switches 111a-n and 126a-n, respectively. Each switch 111a-n and 126a-n has a first switch terminal A connected to one of the contacts of the IS-1 connector 30 and a second switch terminal B connected to one of the lead terminals of the lead connector 40. A control terminal C controls the opening and closing of each switch upon receipt of a control signal. The control terminals C of the switches 111a-n of the pacing multiplexer 110 are connected to the pacing control circuit 115. The pacing control circuit 115 is connected to the program input circuit 80 to receive a programming signal designating, which of the switches 111a-n are closed to connect the pacing set of electrodes through the adapter 100 to pulse generator 20 to receive the pacing signal. The pacing control circuit 115 transfers the appropriate control signals to the control terminals C to close the designated switches 111a-n connected to the pacing electrodes during the period when the pacing signal is active.

The control terminals C of the switches 126a-n of the sensing multiplexer 125 are connected to the sensing control circuit 120. The sensing control terminals of the switches 126a-n of the sensing multiplexer 125 are connected to the sensing control circuit 120. The sensing control circuit 120 is connected to the program input circuit 80 to receive a programming signal designating, which of the switches 126a-n are to be closed to connect the sensing set of electrodes through the adapter 100 of this invention to the pacemaker generator 20 to provide the sense points for the pacemaker generator 20 to sense the heart activity. The sensing control circuit 120 transfers the appropriate control signals to the control terminals C of the sensing multiplexer 125. To close the designated switches 111a-n connected to the sensing electrodes during the period when the pacing signal is inactive and the pulse generator 20 is sensing the heart activity.

The pacing control circuit 115 and the sensing control circuit 120 are connected to the contacts of the IS-1 connector 30. The pacing control circuit 115 and the sensing control circuit 120 examine the IS-1 connector 30 for the presence of the pacing signal. At the beginning of the pacing signal, the pacing control circuit 115 sends a close signal to the respective control terminals C of the pacing multiplexer 110 to cause closure of the selected switches such that the selected pacing electrodes of the lead 5 receive the pacing signal. Moreover at the beginning of the pacing signal, the sensing control circuit 120 sends an open signal to open to the control terminals to cause all the switches of the sensing multiplexer 125 to prevent the pacing pulse from being coupled to the sensing electrodes of the multi-electrode cardiac lead and to avoid frying the sense arcuitry within the pacing electrode.

After the pacing signal has terminated, control circuit 115 sends an open signal to the control terminals to cause all the switches of the pacing multiplexer 110 to be opened. At this same time the sensing control circuit 120 sends a close signal to the appropriate control terminals of the sensing multiplexer 120 to cause closure of the switches connected to the sensing electrodes of the leads to connect the selected sensing electrodes to the IS-1 connector 30.

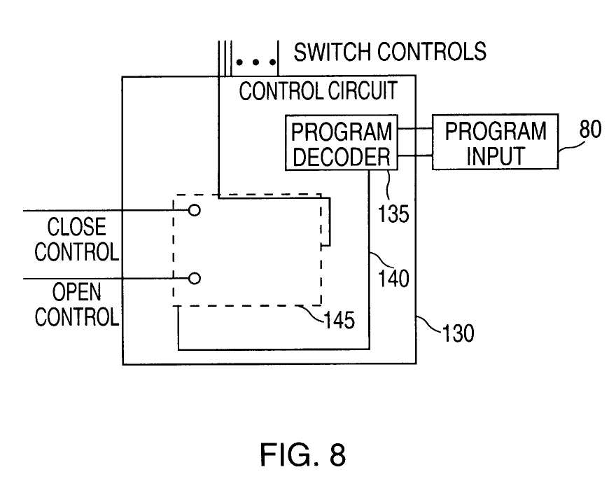

FIG. 8 illustrates an implementation of the pacing control circuit 115 and the sensing control 120 in the form of a control circuit 130. The control circuit 130 has a program decoder 135 that is connected to the program input 80 to receive the programming signal. The program decoder sends the control signal 140 to the logic circuit 145 pulse. The program decoder enables each of the switches (or gates) of the controller. The pacing controller closes the enabled switches on a pacing pulse. The sensing controller opens the enabled switches on a pacing pulse

All electronic embodiments should have a back-up fail-safe mechanism in the switch controller that assures that during a failure the adapter 10, 100 leaves the proper pacing and sensing group of electrodes of the multi-electrode cardiac lead connected to the IS-1 connector 30. The group of electrodes that are connected would be programmed from the programming device, eliminating the possibility that the adapter would route pacing signals to an ineffective pair of electrodes.

The switches 111a-n and 126a-n of the mul1tiplexer 65 of FIG. 5, the pacing multiplexer 110 of FIG. 7 and the sensing multiplexer 120 of FIG. 7 may be implemented as solid state relays that are field effect transistors FET's configured as pass-gates or transmission gates as is known in the art.

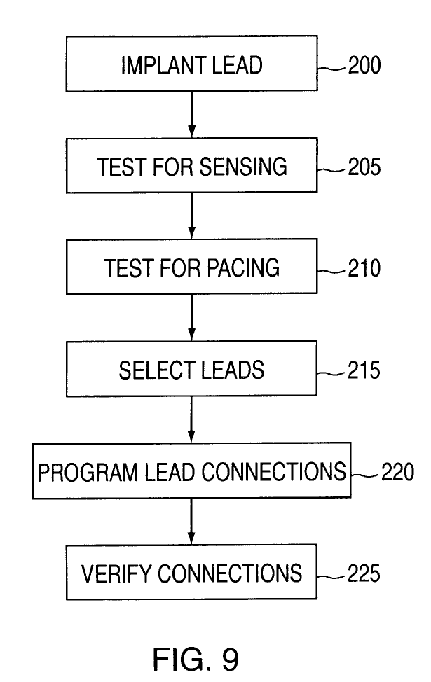

Refer now to FIG. 9 for a description of the steps of the method to select the group of electrodes of the cardiac lead for connection to the IS-1 connector of a pacing pulse generator.

As can be seen from the above description, the adapter (10,100) can be provided in a number of different configurations. In the simplest configuration (FIGS. 3b, 3c, 4) the links of the adapter are set or “burned in” during the implantation procedure. For the other embodiments, (FIG. 5) the links of the multiplexer can be closed and opened at will. Finally in th embodiments of FIGS. 7 and 8 the adapter is dynamic in the sense that it opens and closes the links of the matrix as the patient's heart is being stimulated. After a multi-electrode load 5 is implanted, its electrodes must be designated for the appropriate functions. The physician can inspect the lead and its electrodes through x-ray or other imaging means and designate the electrodes on his own. Alternatively, an automated procedure may be used to identify and designate the electrodes as follows.

The lead 5 is implanted (step 200) into the heart. The lead 5 contains any number of independent electrodes. In the preferred embodiment the multi-electrode cardiac lead may have up to 128 electrodes or even 256 electrodes. Each electrode on the lead is theoretically capable of sensing the heart's electrical activity and delivering an electrical pulse to the heart. The delivery of therapy can be for optimized for bradycardia pacing and for multi-site stimulation for congestive heart failure. The endocardial cardiac lead 5a is placed in one or more chambers of the heart and the epicardial cardiac 5b is placed on the exterior surface of the heart, thus allowing complete sensing and stimulating control of the entire chamber. Alternately, electrodes are placed along the ventricular septum and up into the right ventricular outflow tract. Electrodes may be placed along one wall of the heart chamber or in the atrium and continue into the ventricle. The electrodes are spaced appropriately on the lead for the intended application.

Upon proper implantation (step 200) of the cardiac lead in the heart, each electrode is tested (step 205) to determine which of the electrodes are positional for optimal sensing of the heart activity.

Single site sensing only attempts to determine whether a cardiac event occurred or not. This is determined by observing the cellular electrical activity that initiates the cardiac contraction. This is the same signal that is observed on a surface electrocardiogram (ECG), except at a more localized level. The surface ECG is a summation of the electrical activity of all of the cells of the heart. Depending on how the electrode is placed, the signal seen by a pacemaker can range between less than 1 mV to greater than 10 mV. Obviously, it is desirable to find the location with the largest signal. Thus, during an implant, a location with a good amplitude sensing signal is determined.

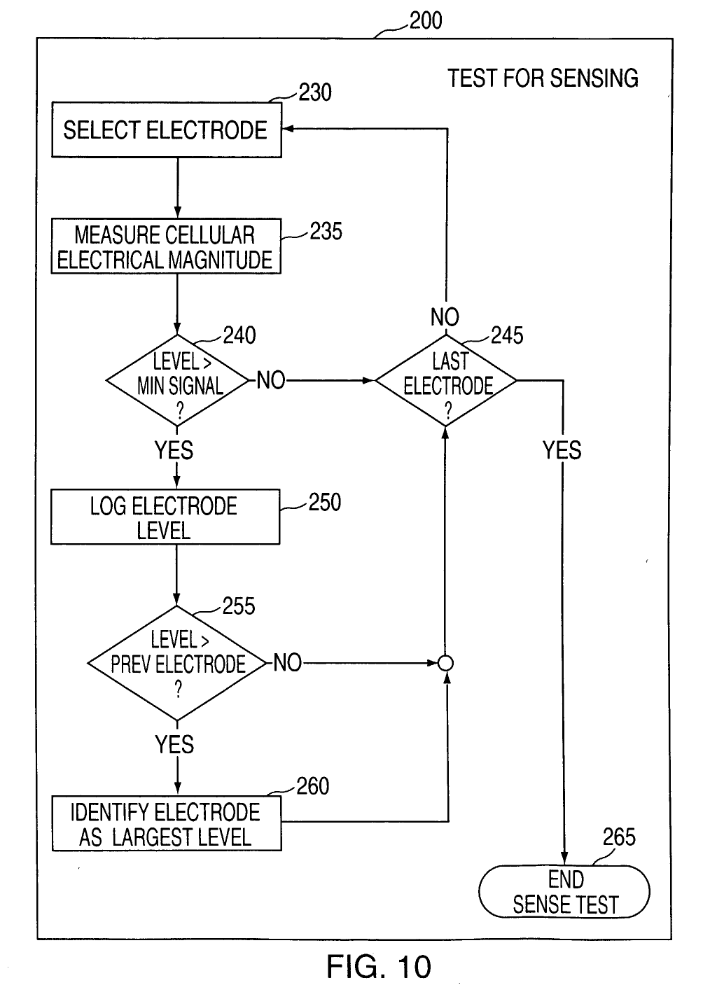

Referring the FIG. 10, an electrode of a cardiac lead is tested as follows. In step 230 one of the electrodes is selected. The magnitude of the intrinsic electrical activity served through the selected ?? is measured (step 235). To be considered for inclusion for sensing, the electrode must provide a sensing signal greater than a minimum signal level. The measured magnitude of the intrinsic electrical activity as sensed by the electrode is compared (step 240) to the minimum acceptable signal level. If the measured signal is not greater than the minimum acceptable signal level, a test if the chosen electrode is the last electrode being tested (step 245) is performed. If it is not the last electrode being tested, a new electrode is selected (step 230).

If the measured magnitude of the intrinsic electrical activity as sensed by the chosen electrode is greater than minimum acceptable signal level, an electrode identifier with the measured level is logged (step 250). The measured magnitude of the intrinsic electrical activity as sensed by the chosen electrode is compared (step 255) to the magnitude as sensed by a previously identified electrode having the maximum measured. If the measured magnitude of the current electrode is not greater than the measured magnitude of the previously identified electrode, the electrode is tested (step 245) for being the last electrode. If the electrode is the last electrode, the sensing testing ends (step 265). If it is not the last electrode, the next electrode is selected (step 230) and tested.

If the measured magnitude of the current electrode is greater than the measured magnitude of the previously identified electrode, the electrode is identified (step 260) as the electrode with the largest magnitude. The electrode is tested (step 245) for being the last electrode. If the electrode is the last electrode, the sensing testing ends (step 265). If it is not the last electrode, the next electrode is selected (step 230) and tested.

Referring back to FIG. 9, each lead is then tested 210 to determine which lead or set of leads are optimally connected for providing the pacing signal to the heart. Using what is referred to in the art as “sweet-spot pacing”, or single-site optimization, pacing is accomplished through only one electrode, but only that electrode that optimizes a desired parameter is chosen.

One parameter that could be optimized is the amount of the cardiac contraction caused by the pacing pulse to a particular electrode. A measure of a good cardiac contraction is the amount of time the entire contraction takes i.e., the QRS width. A wider QRS indicates a slower spread of the wavefront across the heart and is usually typical of a poorly synchronized heartbeat. By pacing through each electrode and measuring the width of the QRS complex, we can find the best site from which to pace the heart.

Other methods, including invasive procedures, could be used to measures of cardiac output to select the optimum site.

Another optimization parameter can be the stimulation threshold, or the provisional amount of energy required to cause the heart to contract from a stimulating pulse (capture). This greatly affects the length of battery life and much time is spent during a pacemaker implant attempting to find the location with the lowest threshold. The threshold is determined by lowering the pacing energy while pacing until the pulses no longer capture the heart. The lowest value that captures the heart and augmented by a safety margin is the threshold. Using the cardiac lead, the threshold of each electrode can be found and pacing is done using the electrode with the lowest threshold.

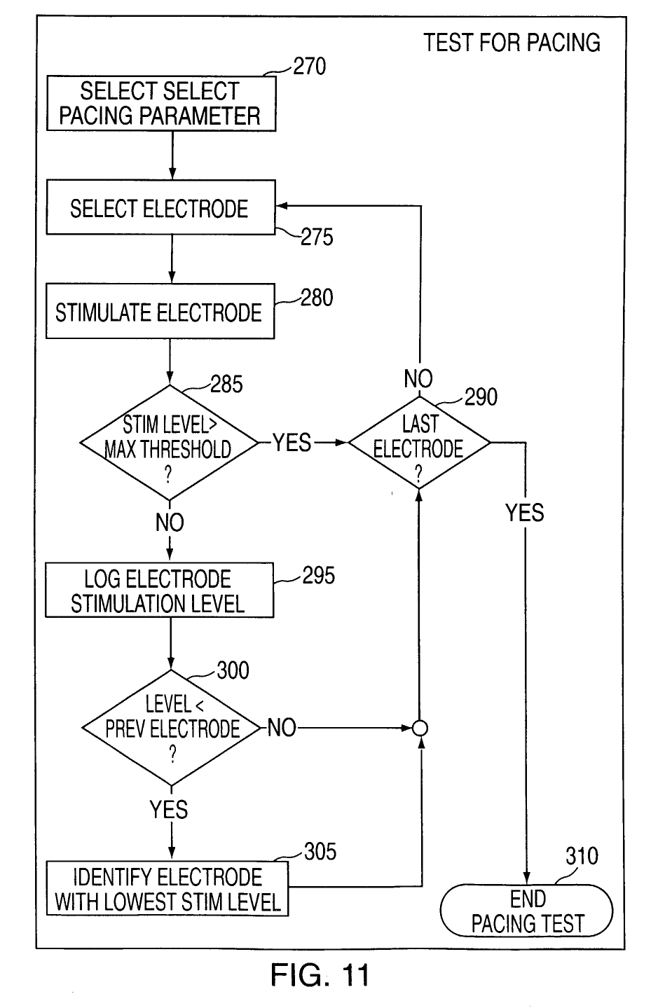

As shown in FIG. 11, the testing (step 210) for pacing begins by selecting (step 270) which parameter is suitable for selecting a cardiac pacing leads. This step may be performed automatically or the parameter may be set by the physician. Next, an electrode of the cardiac lead is chosen (step 275) for testing.

The initial selection (step 275) of the electrode may be random. The electrode most likely to provide the best pacing such as one electrode near the tip of the cardiac lead, or a first terminal location on the connector. As is apparent, any initial choice (step 275) of the electrode is in keeping with the intent of this invention. Further, any pattern of selection of choosing (step 275) subsequent electrodes is also in keeping with the intent of this invention.

The pacing signal is applied (step 280) through the respective electrode to the heart. The stimulation level required to stimulate the heart is recorded and compared (step 285) to a maximum stimulation level allowed. If the stimulation level of the pacing signal is greater than the maximum stimulation level allowed,.the electrode is to be ignored. The electrode is tested (step 290) to determine if it is the last electrode in the cardiac lead to be evaluated. If it is not the last electrode in the multi-electrode cardiac lead to be evaluated, the next electrode is selected (step 275) for testing. If it is the last electrode to be evaluated, the pacing testing ends (step 310).

If the stimulation level of the pacing signal is less that the maximum stimulation level allowed, the electrode identification and the stimulation level is logged (step 295) and compared (step 300) to the stimulation level of the previously identified electrode as having the minimum stimulation level. If the currently tested electrode has a stimulation level greater than the stimulation level of the previously electrode identified as having the minimum stimulation level, the electrode is tested (step 290) if it is the last electrode in the multi-electrode cardiac lead to be evaluated. If it is not the last electrode in the multi-electrode cardiac lead to be evaluated, the next electrode is selected (step 275) for testing. If it is the last electrode to be evaluated, the pacing testing ends (step 310).

If the currently tested electrode has a stimulation level less than the stimulation level of the previously electrode identified as having the minimum stimulation level, the currently tested electrode is identified (step 305) as the electrode having the minimum stimulation level. The electrode is tested (step 290) if it is the last electrode in the multi-electrode cardiac lead to be evaluated. If it is not the last electrode in the multi-electrode cardiac lead to be evaluated, the next electrode is selected (step 275) for testing. If it is the last electrode to be evaluated, the pacing testing ends (step 310).

Once the sensing electrodes and pacing electrodes are determined, the correct combination of sensing electrodes and pacing electrodes are selected (step 215) to be connected to the pacemaker.

If the configurations of FIGS. 3-5 are used, then a compromise between the pacing threshold and the sensing signal must be made in choosing which of the electrodes are to be connected to the pacemaker. The optimization criteria for sensing is simply the site with the combination of the largest sense signal and the lowest stimulation threshold.

The ability to activate the pacing electrode only during pacing and to activate the same electrode during sensing as described for FIG. 7 above eliminates the need for this compromise and can both decrease the implant time and improve the efficacy and reliability of the therapy.

Returning to FIG. 9, after the sensing and pacing electrodes have been designated, the proximal end of lead 5 is inserted into the lead connector 40 of the adapter 10. The desired group of electrodes that provide optimum sensing and pacing are programmed (step 220) within the multiplexer as described above. In other words, the multiplexer is programmed to connect the sense and pace electrodes of lead 5 to the corresponding terminals of the pacemaker 20.

The adapter 10,100 is connected to the IS-1 connector 15 of the pacemaker 20. The functioning of the pacemaker and the programming (step 220) of the multiplexer of the adapter is verified (step 225) for proper operation. The verification may be as simple as observation of the operation of the pacemaker using normal ECG criteria. Alternately, in a pacemaker system having the ability to communicate the status of the connections, the address of the adapter with a coding of the electrodes connected and not connected for comparison to the logging of the sense signal magnitude and the stimulation level logging. This comparison allows for verification and diagnostics of the performance of the pacemaker.

In the procedure set forth in FIG. 9, the adapter is connected to the lead 5 only after the designation of the electrode. The adapter can be connected to the lead right after the implantation, and an external programmer can be connected to the adapter using a standard S1 connector. In this way the programmer can use the adapter to step through the electrodes of lead 5 for scanning, pacing, etc. For example, as shown in FIG. 1a, cable 12 can be temporarily connected to an external programmer 77 as shown. The programmer performs the function as described in FIGS. 9-11 to designate the electrodes, or to provide guidance to a physician regarding the designation of the electrodes. The programmer also sets the links of the adapter based either on the results of the automatic designation, or as requested by the physician.

While this invention has been particularly shown and described with reference to the preferred embodiments thereof, particularly implantable pacemakers, it will be understood by those skilled in the art that various changes in form and details such as use with other cardiac devices such as an implantable cardioverter/defibrillator or ICD may be made without departing from the spirit and scope of the invention.

The invention claimed is:

1. An adapter for connection between a cardiac lead having a distal end including a plurality of electrodes adapted to be associated with a heart of a patient and a proximal end, and a cardiac pacing device, said adapter comprising:
a first connector that receives said proximal end;
a second connector for connection to said cardiac pacing device and having contacts; and
a multiplexer having an input connected to said first connector, an output, and a selector connected to the first connector to couple a group of electrodes of said cardiac lead to said contacts through said output.

2. The adapter of claim 1 wherein said selector comprises:
a plurality of links, each link being connected said input and said output;

3. The adapter of claim 2 wherein said links are permanently settable in one of an open and closed position;.

4. The adapter of claim 3 wherein said links are fusible.

5. The adapter of claim 4 wherein said fusible links are responsive to an electrical signal.

6. The adapter of claim 5 wherein said links close in response to said electrical signal.

7. The adapter of claim 3 wherein said links are breakable.

8. The adapter of claim 7 wherein said links open in response to an electric signal.

9. The adapter of claim 1 wherein the selector comprises a plurality of electric switches, wherein each switch has a first terminal connected to an electrode of said cardiac lead, a second terminal connected to a contact of said second connector, and control terminal that is responsive to a control signal to selectively open and close the respective switch.

10. The adapter of claim 9 wherein said switches are FET transmission gates.

11. The adapter of claim 10 further comprising a control circuit connected to the control terminal of each switch to provide said control signals.

12. The adapter of claim 11 further comprising a programming interface connected to the control circuit to provide an encoded programming signal indicating which electrodes are to be connected to which contacts, said control circuit being adapted to decode said encoded programing signal to produce said control signal.

13. The adapter of claim 12 wherein said programming interface comprises:
a radio frequency receiver attached to said control circuit to receive
said encoded programming signal from a remote radio frequency transmitter.

14. The adapter of claim 1 wherein said multiplexer is electrically operated.

15. The adapter of claim 14 further comprising a power conversion circuit to convert a portion of the energy present in a stimulation pulse provided by the cardiac pacing device to a voltage to power said multiplexer.

16. An adapter of claim 15 wherein the power conversion circuit comprises a battery to provide backup power.

17. The adapter of claim 15 wherein the power conversion circuit comprises a capacitor to receive and retain said energy from the pacing pulse.

18. The adapter of claim 17 wherein the power conversion circuit further comprises:
a first diode connected between a pacing contact of the second connector and the multiplexer circuit to prevent power from being fed to the cardiac pacing device, while allowing said pacing pulse to be received by the capacitor; and
a second diode connected between the battery and the multiplexer to prevent the pacing pulse from interacting with said battery.

19. An adapter connected between a cardiac lead having a distal end including a plurality of electrodes adapted to contact a patient's heart and a proximal end having plurality of terminals, each terminal being connected to one electrode, and a cardiac stimulation device capable of functioning with less electrodes than the number of electrodes in said lead and including a sensing and a stimulation contact, said adapter comprises:
a first connector that receives said plurality of connector terminals of the cardiac lead;
a first multiplexer having an input connected to said first connector, a selector connected to the input to couple a first group of electrodes of said cardiac lead, and an output to transfer sensing signals from said first group of electrodes;
a second multiplexer having an input connected to said first connector,
a selector connected to said input to couple a second group of electrodes of said cardiac lead, and an output to transfer pacing signals to said second group of electrodes; and
a second connector having contacts connected to said cardiac stimulation device and said output of said multiplexer to transfer said sensing signals and the pacing signals between said groups of electrodes and said cardiac pacing device.

20. The adapter of claim 19 further comprising a control circuit connected to selectors to operate said multiplexes.

21. The adapter of claim 20 wherein said control circuit includes a sense control circuit adapted to sense the absence of a stimulation pulse from said cardiac stimulation device to operate said first multiplexer.

22. The adapter of claim 20 wherein said control circuit includes a pacing control circuit connected to said second connector to sense the presence of a stimulation pulse from the cardiac stimulation device to deactivate the first multiplexer and activate the second multiplexer to connect the second group of electrodes to said contacts to allow said stimulation pulse to be transferred to said second group of electrodes.

23. The adapter of claim 19 wherein said selectors each comprise a plurality of switches, each switch has a first switch terminal connected to a connector terminal, a second switch terminal connected to a contact, and control terminal that receives a control signal that causes the first switch terminal to be connected to the second switch terminal.

24. The adapter of claim 23 wherein the switches are FET transmission gates.

25. The adapter of claim 19 further comprising a programming interface connected to said control circuit to provide an encoded programming signal indicating which electrodes are to be connected to said contacts.

26. The adapter of claim 25 wherein said programming interface comprises:
a radio frequency receiver attached to said control circuit to receive said encoded programming signal as a radio frequency transmission from a radio frequency transmitter remote from said hear.

27. The adapter of claim 19 further comprising a power conversion circuit connected to one of said contacts to convert a portion of the energy present in a stimulation pulse provided by the cardiac stimulation device to a voltage to power said adapter.

28. The adapter of claim 27 wherein the power conversion circuit comprises a battery connected to the multiplexer to provide backup power voltage to power the multiplexer in the absence of a stimulator pulse.

29. The adapter of claim 28 wherein the power conversion circuit further comprises a capacitor in communication with said one contact to receive and retain said energy from said stimulation.

30. The adapter of claim 29 wherein the power conversion circuit further comprises:
a first diode connected between said one contact and the multiplexer circuit to prevent voltage of said battery from being fed to said cardiac stimulation device, while allowing said stimulation pulse to be received by the capacitor; and
a second diode connected between said battery and said multiplexer to prevent said stimulation pulse from interacting with said battery.

31. A cardiac pacing system comprising:
a cardiac lead having a distal end including a plurality of electrodes adaptable to be implanted within a heart and a proximal end having plurality of lead terminals, each lead terminal being connected to one of said electrodes;
a cardiac stimulation device capable of receiving and functioning with a less number of electrodes than the electrodes in said lead; and
an adapter connected between said cardiac lead and said cardiac stimulation device, said adapter including:
a first connector that mates with said terminals;
a multiplexer having an input connected to said first connector, a selector connected to said input to chose a group of electrodes, and an output; and
a second connector compatible with and connected to said cardiac stimulation device; and
being connected to said output to transfer said sensing signals and said stimulation signals between the chosen group of electrodes and the cardiac stimulation device.

32. The cardiac pacing system of claim 31 wherein said selector comprises:
a plurality of links, each link being connected between each one of said terminals, and
a link programmer to activate one of said links to selectively connect a respective terminal uniquely with one of said contacts.

33. The cardiac pacing system of claim 32 wherein said link programmer selectively generates a voltage and wherein said links are fusible links, which, in response to said voltage are fused to form a closed electrical path.

34. The cardiac pacing system of claim 32 wherein said links are breakable links, wherein said programmer is adapted to generate high current, and wherein said breakable links are responsive to said current to break said connection.

35. The cardiac pacing system of claim 31 wherein said selector comprises a plurality of switches, each switch has a first switch terminal connected to a lead terminal, a second switch terminal connected to one of said contacts, and control terminal that receives a control signal that causes said first terminal switch to be connected to the second switch terminal.

36. The cardiac pacing system of claim 35 wherein the switches are FET transmission gates.

37. The cardiac pacing system of claim 35 wherein the adapter further comprises a control circuit connected to the control terminal of each switch to provide said control signals to selectively activate said switches.

38. The cardiac pacing system of claim 37 wherein said adapter further comprises a programming interface connected to the control circuit to provide an encoded programming signal indicating which electrodes are to be connected to which contacts, and wherein said control circuit decodes said encoded programming signal to form the control signal.

39. The cardiac pacing system of claim 38 wherein said programming interface comprises:
a radio frequency receiver attached to said control circuit to receive said encoded programming signal as a radio frequency transmission from a remote radio frequency transmitter.

40. The cardiac pacing system of claim 31 wherein said adapter further comprises a power conversion circuit to provide a voltage to power said multiplexer.

41. The cardiac pacing system of claim 40 wherein said power conversion circuit comprises a battery connected to said multiplexer to provide said voltage in the prolonged absence of said pacing pulses.

42. The cardiac pacing system of claim 41 wherein said power conversion circuit further comprises a capacitor in communication with a pacing contact of the contacts to receive and retain said energy from said pacing pulse.

43. The cardiac pacing system of claim 42 wherein the power conversion circuit further comprises:
a first diode connected between said pacing contact and said multiplexer circuit to prevent voltage from said battery from being fed to said cardiac pacing device, while allowing said pacing pulse to be received by said capacitor; and
a second diode connected between said battery and the multiplexer to prevent said pacing pulse from interacting with said battery.

44. A cardiac pacing system comprising:
a cardiac lead having a distal end including a plurality of implantable electrodes and a proximal end having plurality of lead terminals, each lead terminal being connected to one electrode;
a cardiac pacing device adapted to function with less cardiac electrodes than the number of electrodes in said lead; and
an adapter connected between said lead and said cardiac pacing device, said adapter including:
a first connector that mates with said lead terminals;
a sensing multiplexer having a sense input connected to said first connector, a sense selector connected to said sense input to chose a first group of electrodes from said plurality of electrodes, and a sense output to transfer sensing signals and signals from said first group of electrodes;
a pace multiplexer having a pace input connected to said first connector, a pace selector connected to said pace input to chose a second group of electrodes from said plurality of electrodes, and a pace output to transfer pacing signals to said second group of electrodes;
a second connector having contacts and being connected to said cardiac pacing device and said pace output to transfer said pacing signals between said groups of electrodes and said cardiac pacing device;
a sense control circuit connected to said first multiplexer and said contacts to sense an absence of a pacing pulse from said cardiac pacing device to activate said first multiplexer to connect said first group of electrodes to said second connector such that said cardiac pacing device can sense intrinsic cardiac activity; and
a pace control circuit connected to said second multiplexer and said contacts of the second connector to sense the presence of said pacing pulse to deactivate said first multiplexer and activate said second multiplexer to connect said second group of electrodes to said contacts to allow said pacing pulse to be applied to said second group of electrodes.

45. The cardiac pacing system of claim 44 wherein selectors each comprise a plurality of switches, whereby each switch has a first switch terminal connected to one lead terminal, a second switch terminal connected to one of said contacts, and a control terminal that is responsive to a control signal that causes the first switch terminal to be connected to the second switch terminal.

46. The cardiac pacing system of claim 45 wherein the switches are FET transmission gates.

47. The cardiac pacing system of claim 46 wherein said adapter further comprises a programming interface connected to said control circuit to provide an encoded programming signal indicating which electrodes are to be connected to which contacts, said control circuit being adapted to decode said encoded programming signal to generate the control signal.

48. The cardiac pacing system of claim 47 wherein said programming interface comprises:
a radio frequency receiver attached to said control circuit to receive said encoded programming signal as a radio frequency transmission from a remote radio frequency transmitter.

49. The cardiac pacing system of claim 45 wherein said adapter further comprises a power conversion circuit connected between said contacts and said multiplexer to convert a portion of the energy present in a pacing pulse provided by said cardiac pacing device to a voltage to power said multiplexer.

50. The cardiac pacing system of claim 49 wherein said power conversion circuit comprises a battery to provide the voltage to power said multiplexer when said cardiac pacing device does not provide said pacing pulse.

51. The cardiac pacing system of claim 50 wherein the power conversion circuit further comprises a capacitor in communication with a pacing contact of the contacts of the second connector to receive and retain said energy from the pacing pulse to power said multiplexer.

52. The cardiac pacing system of claim 51 wherein the power conversion circuit further comprises:
a first diode connected between a pacing contact of the second connector and said multiplexer circuit to prevent voltage from the battery from being fed to said cardiac pacing device, while allowing said pacing pulse to be received by said capacitor; and
a second diode connected between said battery and said multiplexer to prevent said pacing pulse from interacting with said battery.

53. A method for selecting electrodes of a cardiac lead to be connected to input contacts of a cardiac stimulation device comprising the steps of:
placing the cardiac lead in contact with a patient's heart;
designating a sense electrode from said electrodes;
designating a pace electrode from said electrodes;
programming an adapter to connect the designated electrodes to the cardiac pacing device; and
connecting said adapter to said cardiac pacing device and said lead.

54. The method of claim 53 wherein said sense electrode is designated by testing said electrodes to detect sensed signals from said electrodes and selecting said sense electrode based on said sensed signals.

55. The method of claim 54 wherein said sense electrode is designated by determining a maximum sensed signal from said sensed signals.

56. The method of claim 53 wherein said pace electrode is designated by determining which pace electrodes are capable of capturing the heart.

57. The method of claim 56 further comprising applying pacing pulses to said electrodes.

58. The method of claim 57 further comprising determining a threshold level associated with each electrode.

59. A cardiac stimulation system comprising:
a plurality of electrodes adapted to contact a patient's heart;
an implantable cardiac stimulating device adapted to sense intrinsic cardiac activity in a patient's heart and to generate stimulation pulses corresponding to said intrinsic cardiac activity; and
an adapter connected to said plurality of electrodes and said cardiac stimulating device, said adapter being constructed and arranged to selectively couple a subset of said plurality of electrodes to said cardiac stimulating device to allow said cardiac stimulating device to sense said cardiac activity and to apply said stimulating pulses.

60. The system of claim 60 wherein said adapter couples a first subset of electrodes to said device for sensing said cardiac activity and a second subset set of electrodes for said stimulating pulser.

61. The system of claim 60 wherein said first set of electrodes is not coupled to said device while said stimulating pulses are applied.
